(19) United States
(12) Patent Application Publication
(10) Pub. No.: US 2018/0235874 A1
(43) Pub. Date: Aug. 23, 2018

(54) Coconut Oil and Honey Throat Lozenge

(71) Applicant: Gina Bassile Hickham, Orlando, FL (US)

(72) Inventor: Gina Bassile Hickham, Orlando, FL (US)

(21) Appl. No.: 15/851,225

(22) Filed: Dec. 21, 2017

Related U.S. Application Data
(60) Provisional application No. 62/441,490, filed on Feb. 21, 2017.

(51) Int. Cl. A61K 9/00; A61K 47/42; A61K 36/889; A61P 11/04; A61K 31/70; A61P 31/04; A61K 35/644

(52) U.S. Cl. CPC A61K 9/0056; A61K 47/42; A61K 36/889; A61K 35/644; A61K 31/70; A61P 31/04; A61P 11/04

(57) ABSTRACT

This organic lozenge may be beneficial to individuals with allergies, sore throat, dry mouth, colds and/or flu based on the many scientific benefits of coconut oil and honey. These benefits include reduced inflammation, anti-bacterial and viral properties, which help fight illness. The physical properties of coconut oil and honey are known to suppress a cough and moisturize the mouth and throat.

### CROSS REFERENCE TO RELATED APPLICATIONS

Not applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

It is requested that this utility patent be issued based on the “composition of matter” which has taken place to create this organic throat lozenge. This lozenge may be beneficial to individuals with allergies, sore throat, dry mouth, colds and/or flu based on the many scientific benefits of coconut oil.

### BRIEF SUMMARY OF THE INVENTION

This organic lozenge may be beneficial to individuals with allergies, sore throat, dry mouth, colds and/or flu based on the many scientific benefits of coconut oil, honey and essential oils.

Coconut oil contains Lauric acid and also has a high level of antioxidants. Because of these properties, it is known to fight bacteria, create a hostile environment for viruses and to reduce inflammation. Also, the texture of the coconut oil is extremely smooth and soothing to a sore throat.

### BRIEF DESCRIPTION OF THE SEVERAL VIEWS OF THE DRAWING

Not applicable

### DETAILED DESCRIPTION OF THE INVENTION

Process for making the Organic Coconut oil and honey throat lozenge—

This lozenge is made from a mixture of the following ingredients—

59.98%—Sugar mixture which is a 40/60 blend of Organic Rice Syrup and Organic Cane Sugar

25%—Organic Expeller Pressed Coconut Oil

12.5%—Organic Honey

2.5%—Sunflower Lecithin (used as an emulsifier)

0.025%—oils of various flavors (i.e. (but not limited to)—lemon, ginger, cinnamon, turmeric, peppermint, etc.)

All of the above ingredients are combined in a cooker and heated to 320 degrees Fahrenheit. The mixture then is hand mixed with the flavor oils on a table, where it begins to harden and cool. It is then put through a machine that cuts it into 4 gram pieces. Once completely cooled, it is ready to be consumed.

How to use the lozenge—put lozenge in mouth until it melts.

1. (canceled)

2. A lozenge comprising coconut oil and honey.

3. The lozenge of claim 2 having a mass of 4 grams.

4. The lozenge of claim 2 containing a minimum of 0.6 grams of coconut oil.

5. The lozenge of claim 2 further comprising organic cane sugar, organic coconut oil, organic honey, organic rice syrup, sunflower lecithin, flavorings and mixtures thereof.

6. The lozenge of claim 2 further comprising a flavoring agent, wherein the flavoring agent is selected from the group consisting of lemon, ginger, turmeric, eucalyptus, mint and strawberry.

7. A method for lubricating the throat which is administered as a sublingual lozenge, the method comprising:
a) formulating the lozenge of claim 2; and
b) administering the lozenge of claim 2 sublingually to a human until the lozenge is dissolved in the mouth of the human.
